(19) United States
(12) Patent Application Publication
(10) Pub. No.: US 2026/0250173 A1
(43) Pub. Date: Aug. 27, 2026

(54) MIXING AND SOLIDS REMOVAL SYSTEM FOR RACETRACK DIGESTER AND RACETRACK DIGESTER INCLUDING SAME

(71) Applicant: MONTAUK ENERGY HOLDINGS, LLC, Pittsburgh, PA (US)

(72) Inventor: BRANDON CHARLES SMITH, Robinson Township, PA (US)

(73) Assignee: MONTAUK ENERGY HOLDINGS, LLC, Pittsburgh, PA (US)

(21) Appl. No.: 19/551,844

(22) Filed: Feb. 27, 2026

Related U.S. Application Data
(60) Provisional application No. 63/764,244, filed on Feb. 27, 2025.

(51) Int. Cl. C02F 11/04; B01F 27/11; B01F 35/71

(52) U.S. Cl. CPC C02F 11/04; B01F 27/11; B01F 35/7176

(57) ABSTRACT

A racetrack digester for use in obtaining biogas from organic materials through the process of anaerobic digestion includes a containment wall disposed about and defining a digestion region structured to retain a volume of the organic materials, a plurality of mixer arrangements spaced along the containment wall, and a plurality of piping systems spaced along the containment wall. The digester further includes a system component associated with a respective piping system of the plurality of piping systems. The system component is one of a mixing system for mixing solids in the digestion region back into suspension or a solids removal system for removing solids from the digestion region.

### CROSS REFERENCE TO RELATED APPLICATIONS

This application claims priority to U.S. Provisional Patent Application Serial No. 63/764,244, filed on February 27, 2025, titled “MIXING AND SOLIDS REMOVAL SYSTEM FOR RACETRACK DIGESTER AND RACETRACK DIGESTER INCLUDING SAME” the disclosure of which is incorporated herein by reference.

### FIELD

The disclosed concept relates generally to digesters for processing waste materials for producing biogas and, more particularly, to racetrack digesters including mixing and solids removal systems. The disclosed concept further relates to mixing and solids removal systems for use in digesters.

### BACKGROUND

Embodiments of the concept(s) disclosed herein improve operations of a digester used in the production of biogas, such as a racetrack digester. As is known, racetrack digesters are used to break down (via anaerobic digestion) organic waste materials from plants and animals (e.g., cow manure) to produce biogas (e.g., methane) and fertilizer materials. An example racetrack digester 10 upon which embodiments of the disclosed concept improve is shown partially schematically in FIG. 1. Such digester 10 includes several fixed mixer arrangements 12 disposed in fixed positions along the containment wall 14 of the digester 10 for moving the organic waste materials 16 within a digestion region 18 of the digester 10. Each fixed mixer arrangement 12 includes a rotatable shaft 20 that extends through the containment wall 14 into the organic waste materials 16 being processed in the digestion region 18. Each fixed mixer arrangement 12 also includes a propeller 22 disposed in the waste material 16 and fixedly coupled to a distal end (not numbered) of each shaft 20 such that when the shaft 20 is rotated (e.g., via a motor or other suitable drive arrangement 24 positioned exterior to the digestion region 18) the propeller 22 spins, thus generally pushing the waste materials 16 about the digestion region 18 of the digester 10 around a center wall 26.

The example digestion region 18 of the conventional digester 10 of FIGS. 1-3 is 150’ wide, 300’ long, and 20’ deep, however, it is to be appreciated that the arrangements/concepts described herein may be applied to digesters of other size/arrangement without varying from the scope of the disclosed concept. During a cleanout of the digestion region 18 of the digester 10, apart from the regions generally immediately adjacent the fixed mixer arrangements 12, the digestion region 18 was almost completely full of sand and un-digestible solids, with the solids nearly piled to the top of the containment wall 14. FIG. 2 is a photo from the cleanout, where the cover 28 of the digester 10 was completely removed, and the extent of the solids accumulation within the digestion region 18 is plainly visible. FIG. 3 is a second photo of the same digester as FIG. 2 taken a couple of months prior to the photo of FIG. 2 with the cover 28 deflated. In FIG. 3, the deflated cover 28 held up in numerous locations, e.g., region 30, by large mounds of sand and other solids below are plainly visible.

One of the most critical parameters for digester operations is the retention time of the materials 16, which is calculated by dividing average daily flow rate by the total volume of the processing region. The anerobic digestion process needs a minimum amount of retention time to fully digest the material to produce biogas. For the racetrack digester 10, that time is around 25-30 days. When the available volume inside the digester begins to be reduced due to the accumulation of solids, the retention time is also reduced, ultimately reducing the biogas production from the digester 10. Without a way to properly manage the solids, the solids eventually take up enough volume to significantly reduce the biogas production to a point at which other operational issues arise and target production can no longer be maintained. At such point, a full cleanout of the digestion region 16 of the digester 10 is needed in order to return to normal operating conditions. Due to the size of the digestion region 18 of such digesters 10 and the construction thereof, the cleanout typically involves finding a way to effectively access the digesting region 18 (e.g., removing all or a portion of the cover 28, cutting away parts of the containment walls 14 for access) and then spending several weeks or months cleaning out the digesting region 18 (e.g., with skid loaders and/or other suitable means), reassembling/rebuilding the digester 10 as needed, and getting the digester 10 back into service. Such cleanout operation not only takes a significant amount of time, time in which biogas is obviously not being produced, but also requires a considerable amount of monetary investment.

Accordingly, a need exists for improved arrangements for maintaining racetrack digesters in operating condition, thus maintaining optimum biogas production for longer periods of time and reducing the frequency and need for costly cleanouts of the digester.

### SUMMARY

Embodiments of the disclosed concept improve upon conventional digesters by providing solutions which decrease the need for cleanouts and thus increase the operation time.

In one example embodiment, a racetrack digester for use in obtaining biogas from organic materials through the process of anaerobic digestion is provided. The racetrack digester comprises: a containment wall disposed about and defining a digestion region structured to retain a volume of the organic materials; a plurality of mixer arrangements spaced along the containment wall, each mixer arrangement structured to move the organic materials within the digestion region and comprising: a rotatable shaft that extends into the digestion region; a propeller coupled to a distal end of the shaft in the digestion region; and a drive arrangement coupled to a proximal end of the shaft opposite the propeller, the drive arrangement structured to rotate the shaft and propeller to move the organic materials about the digestion region; a plurality of piping systems spaced along the containment wall, each piping system comprising: a first piping arrangement defining a first conduit extending between a first end positioned at or about a top of the containment wall outside of the digestion region and a second end positioned at or about a floor of the containment region; and a second piping arrangement defining a second conduit extending between a first end positioned at or about a top of the containment wall outside of the digestion region and a second end positioned at or near a normal liquid level within the digestion region; and a system component associated with a respective piping system of the plurality of piping systems, wherein the system component comprises one of: a mixing system comprising a mobile chopper pump having a suction inlet in fluid communication with the first end of the second conduit defined by the second piping arrangement of the respective piping system of the plurality of piping systems and a discharge outlet in fluid communication with the first end of the first conduit defined by the first piping arrangement of the respective piping system; or a solids removal system comprising: a mobile chopper pump having a suction inlet in fluid communication with the first end of the first conduit defined by the first piping arrangement of the respective piping system of the plurality of piping systems and a discharge outlet; and a solids separation system having an inlet in fluid communication with the discharge outlet of the mobile chopper pump and an outlet in fluid communication with the first end of the second conduit defined by the second piping arrangement of the respective piping system.

Each piping system of the plurality of piping systems may further comprise a first isolation valve coupled at the first end of the first conduit and a second isolation valve coupled at the first end of the second conduit, wherein the first isolation valve is positioned and structured to selectively control passage of fluid through the first conduit, and wherein the second isolation valve is positioned and structured to selectively control passage of fluid through the second conduit.

The plurality of mixer arrangements and the plurality of piping systems may be spaced along the containment wall such that at least one piping system is disposed between any two adjacent mixer arrangements.

The plurality of mixer arrangements and the plurality of piping systems may be spaced along the containment wall such that at least two piping systems of the plurality of piping systems are disposed between at least two adjacent mixer arrangements of the plurality of mixer arrangements.

The first piping arrangement may comprise: a straight first portion that extends through the containment wall; a straight end portion positioned near the floor of the digestion region; and a straight intermediate portion that extends between the first portion and the end portion and is coupled with each of the first portion and the end portion via elbows. The elbows may comprise forty-five degree elbows. The second piping arrangement may comprise a single straight portion that extends through the containment wall. The second piping arrangement may comprise a single straight portion that extends through the containment wall.

The plurality of mixer arrangements may comprise at least seven mixer arrangements, and the plurality of piping systems may comprise at least eight piping systems.

The racetrack digester may further comprise another system component associated with another respective piping system of the plurality of piping systems. The system component may comprise the mixing system and the other system component may comprise another mixing system. The system component may comprise the mixing system and the other system component may comprise the solids removal system.

Each mixer arrangement may be disposed in a fixed position along the containment wall and wherein the rotatable shaft of each mixer arrangement extends through the containment wall.

Such improvements and other objects, features, and characteristics of the disclosed concept, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are provided for the purpose of illustration and description only and are not intended as a definition of the limits of the disclosed concept.

### BRIEF DESCRIPTION OF THE DRAWINGS

A full understanding of the disclosed concept can be gained from the following description of the preferred embodiments when read in conjunction with the accompanying drawings in which:

FIG. 1 is a partially schematic plan view of a conventional racetrack digester upon which embodiments of the disclosed concept improve;

FIG. 2 is a photo of a conventional racetrack digester with the top cover removed prior to a cleanout;

FIG. 3 is another photo of the racetrack digester of FIG. 3 with the top cover in place but deflated taken a couple months prior to the photo of FIG. 2;

FIG. 4 is a partially schematic plan view of a racetrack digester in accordance with an example embodiment of the disclosed concept;

FIG. 5 is a partially schematic elevation view of a piping arrangement installed in a portion of a racetrack digester shown with some components of a mixing and solids removal system in accordance with an example embodiment of the disclosed concept shown in a mixing mode; and

FIG. 5 is a partially schematic elevation view of a mixing and solids removal system having a pipe system installed in a portion of a racetrack digester in accordance with an example embodiment of the disclosed concept shown in a mixing mode; and

FIG. 6 is a partially schematic elevation view of a mixing and solids removal system having a pipe system installed in a portion of a racetrack digester in accordance with an example embodiment of the disclosed concept shown in a solids removal mode.

### DETAILED DESCRIPTION

It will be appreciated that the specific elements illustrated in the figures herein and described in the following specification are simply exemplary embodiments of the disclosed concept, which are provided as non-limiting examples solely for the purpose of illustration. Therefore, specific dimensions, orientations, assembly, number of components used, embodiment configurations and other physical characteristics related to the embodiments disclosed herein are not to be considered limiting on the scope of the disclosed concept.

Directional phrases used herein, such as, for example, clockwise, counterclockwise, left, right, top, bottom, upwards, downwards and derivatives thereof, relate to the orientation of the elements shown in the drawings and are not limiting upon the claims unless expressly recited therein.

As used herein, the singular form of “a”, “an”, and “the” include plural references unless the context clearly dictates otherwise.

As used herein, “structured to [verb]” means that the identified element or assembly has a structure that is shaped, sized, disposed, coupled and/or configured to perform the identified verb. For example, a member that is “structured to move” is movably coupled to another element and includes elements that cause the member to move or the member is otherwise configured to move in response to other elements or assemblies. As such, and as used herein, “structured to [verb]” recites structure and not function. Further, as used herein, “structured to [verb]” means that the identified element or assembly is intended to, and is designed to, perform the identified verb. Thus, an element that is merely capable of performing the identified verb but which is not intended to, and is not designed to, perform the identified verb is not “structured to [verb].”

As used herein, the statement that two or more parts or components are “coupled” shall mean that the parts are joined or operate together either directly or indirectly, i.e., through one or more intermediate parts or components, so long as a link occurs. As used herein, “directly coupled” means that two elements are directly in contact with each other. As used herein, “fixedly coupled” or “fixed” means that two components are coupled so as to move as one while maintaining a constant orientation relative to each other. Accordingly, when two elements are coupled, all portions of those elements are coupled. A description, however, of a specific portion of a first element being coupled to a second element, e.g., an axle first end being coupled to a first wheel, means that the specific portion of the first element is disposed closer to the second element than the other portions thereof. Further, an object resting on another object held in place only by gravity is not “coupled” to the lower object unless the upper object is otherwise maintained substantially in place. That is, for example, a book on a table is not coupled thereto, but a book glued to a table is coupled thereto.

As used herein, the phrase “removably coupled” or “temporarily coupled” means that one component is coupled with another component in an essentially temporary manner. That is, the two components are coupled in such a way that the joining or separation of the components is easy and would not damage the components.

As used herein, the term “number” shall mean one or an integer greater than one (i.e., a plurality). Thus, for example, a “number of elements” means one element or a plurality of elements.

Embodiments of the disclosed concept address shortcomings of conventional racetrack digesters by utilizing mixing and solids removal systems and related components positioned in selective locations of the digester (typically in areas along the perimeter of the digester) where solids accumulation has historically been problematic (e.g., inefficient mixing zones between mixers) to reduce the amount of settled solids that accumulate within the digestion region 18 of a digester, such as the racetrack digester 10 previously discussed, and significantly extend the time between performing costly, full-scale cleanouts of the digestion region 18. Systems in accordance with example embodiments of the disclosed concept have two primary functions: 1) provide supplemental mixing at and about the floor of the digestion region to resuspend settled solids, allowing them to continue moving through the digestion region and eventually exit via an effluent stream, and 2) provide a means to remove settled solids from the bottom of the digestion region, separate and remove the solids externally to the digestion region, and reintroduce the clean liquid stream back into the digestion region for continued processing.

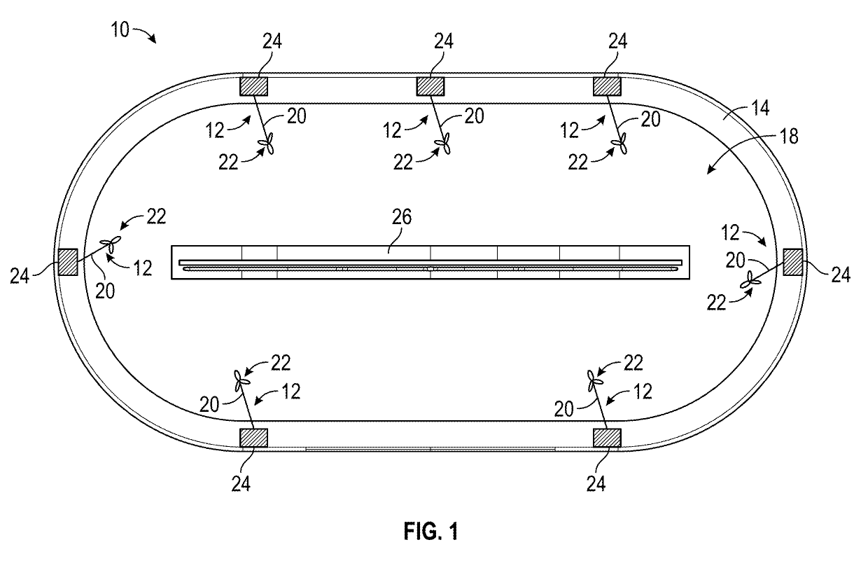

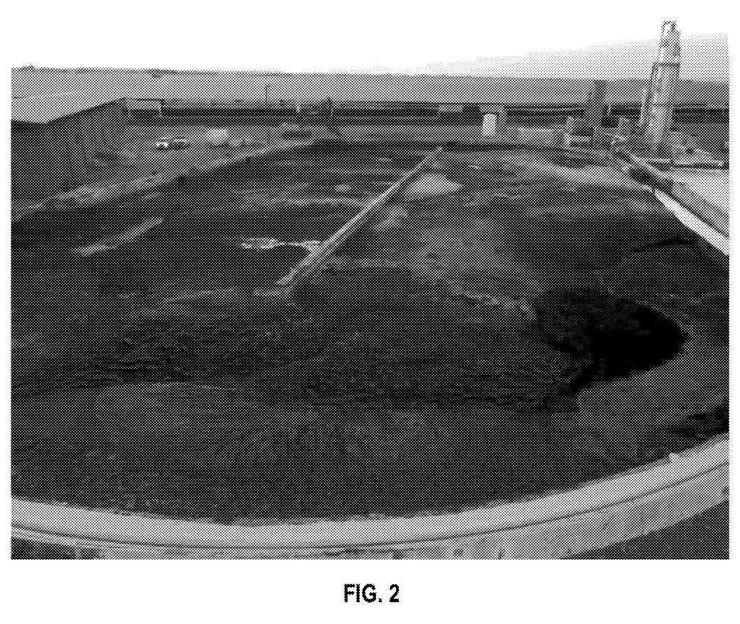

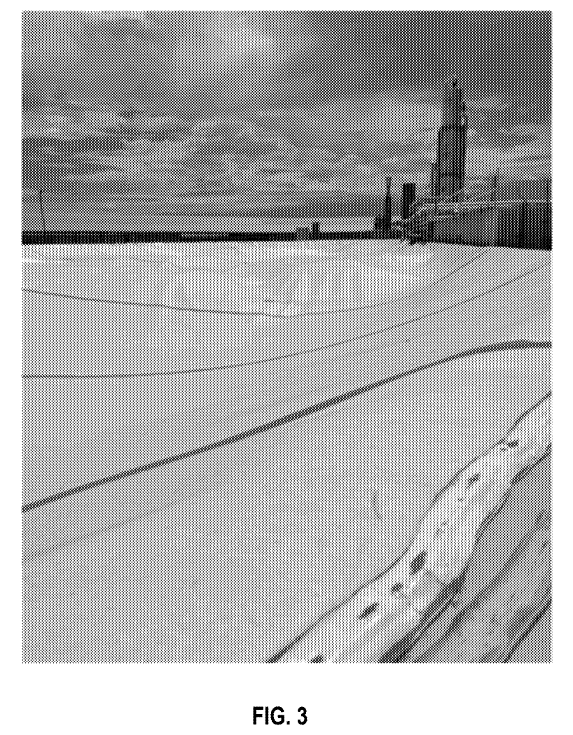

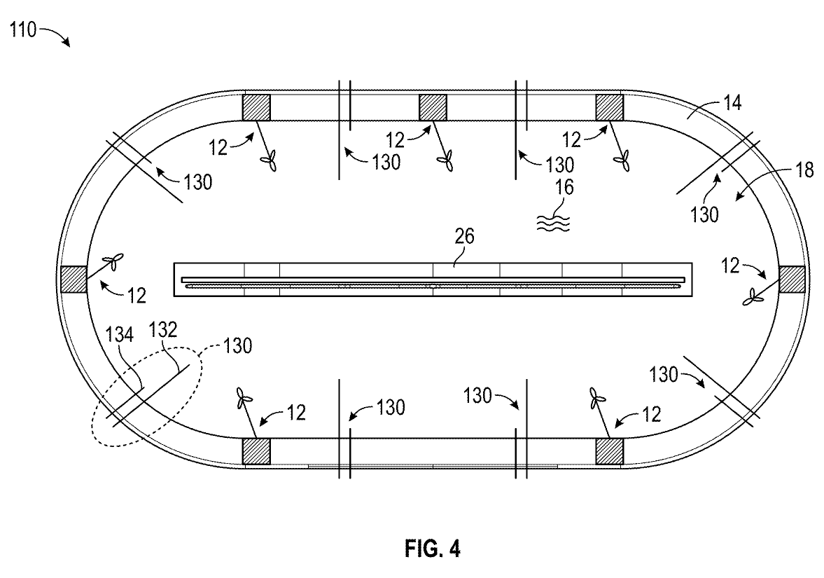

A partially schematic plan view of a racetrack digester 110 in accordance with an example embodiment of the disclosed concept is shown in FIG. 4. The racetrack digester 110 is of the same construction and includes the same components previously discussed in regard to the racetrack digester 10 shown in FIG. 1, and additionally includes new components that provide, inter alia, the improvements noted above.

Referring to FIG. 4, in addition to the elements previously discussed in regard to the racetrack digester 10, the racetrack digester 110 further includes a plurality of piping systems 130 spaced along the containment wall 14. In the example digester 110 shown in FIG. 4, the plurality of piping systems 130 comprises eight piping systems 130 spaced roughly equally about the containment wall 14 and generally spaced evenly among the mixer arrangements 12, however, it is to be appreciated that one or more of the quantity and/or locations of the pipe systems 130 may be varied without varying from the scope of the disclosed concept.

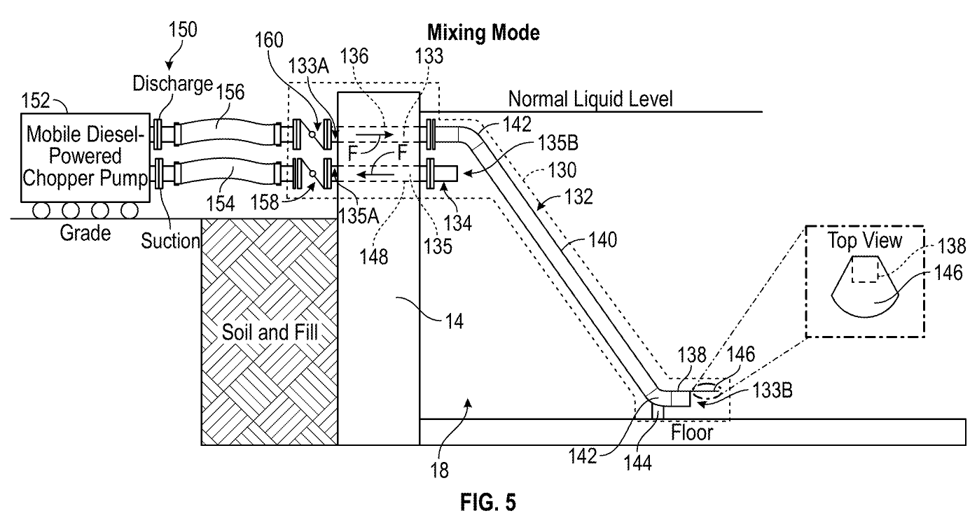

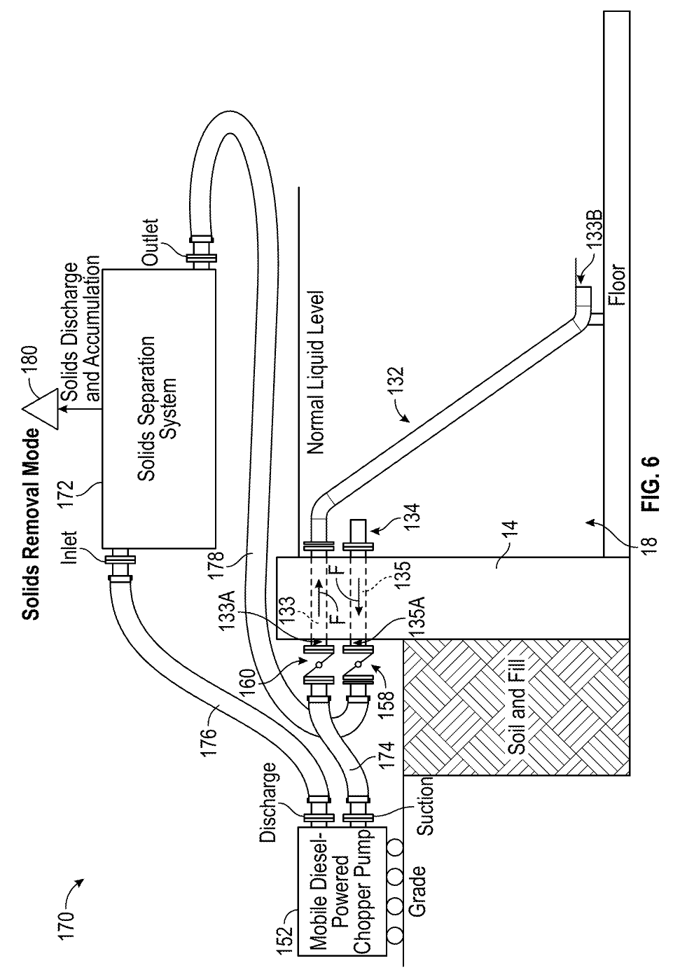

Continuing to refer to FIG. 4 and additionally FIGS. 5 and 6, each piping system 130 includes a first piping arrangement 132 and a second piping arrangement 134. The first piping arrangement 132 defines a first conduit 133 that extends between a first end 133A positioned at or about a top of the containment wall 14 outside of the digestion region 18 and a second end 133B positioned at or about a floor of the containment region 18. The second piping arrangement 134 defines a second conduit 135 extending between a first end 135A positioned at or about a top of the containment wall 14 outside of the digestion region 18 and a second end positioned 135B below, but typically near, the normal liquid level within the digestion region 18.

In the example embodiment shown in FIGS. 5 and 6, the first piping arrangement 132 includes a first portion 136 that extends through the containment wall 14, an end portion 138 positioned near the floor of the digestion region 18, and an intermediate portion 140 that extends between the first portion 136 and the end portion 138 and is coupled with each via elbows 142. In such example embodiment, the elbows 142 are 45 degree elbows, however, it is to be appreciated that elbows 142 of other angles may be employed without varying from the scope of the disclosed concept. In such example, the end portion 138 is spaced a distance (not numbered) above the floor of the digestion region 18 by a support 144 such that the second end 133B of the first conduit 133 is disposed above the floor (e.g., without limitation, about 24 inches in such example). In such embodiment, a deflection plate 146 is provided above the second opening 133B to selectively direct material discharged from the second opening. Meanwhile, in such example embodiment the second pipe arrangement 134 generally includes a single straight portion 148 that extends straight through the containment wall 14.

In addition to the aforementioned elements, the racetrack digester 110 further includes one or more system components for carrying out the mixing and/or removing of solids within the digestion region 18. Such a system component in the form of a mixing system 150 in accordance with an example embodiment of the disclosed concept is shown in FIG. 5 connected with a respective piping system 130 of the plurality of piping systems 130. The mixing system 150 comprises a mobile chopper pump 152 (e.g., without limitation, diesel powered). As used herein, a “mobile chopper pump” is a pump that is designed to macerate tough solids – such as organic debris - before it enters the impeller, and that is mobile (i.e., readily moveable from one location to another).

Continuing to refer to FIG. 5, the mobile chopper pump 152 includes a suction inlet that, when in the mixing mode such as shown in FIG. 5, is in fluid communication with the first end 135A of the second conduit 135 defined by the second piping arrangement 134 of the respective piping system 130 of the plurality of piping systems 130. The mobile chopper pump 152 further has a discharge outlet in fluid communication with the first end 133A of the first conduit 133 defined by the first piping arrangement 132 of the respective piping system 130. In such example, the connections between the inlet and outlet of the mobile chopper pump 152 and the first ends 135A and 133A are made via flexible hoses 154 and 156 and isolation valves 158 and 160. Once the mobile chopper pump 152 is connected, the isolation valves 158 and 160 can be opened and the chopper pump 152 turned on. The pump suction pulls liquid from the upper section of the liquid in the digestion region 18 and discharges it to the bottom of the digestion region 18. As the liquid exits the discharge pipe (i.e., the second end 133B of the first conduit 133) at the bottom of the digestion region 18, the force of the discharged liquid mixes the settled solids, bringing them back into suspension. Arrows F show the direction of flow within the piping system 130 of such example. Once the chopper pump 152 has been running for a certain period of time, it can be turned off, the isolation valves 158 and 160 closed, and the chopper pump 152 disconnected and moved to the next piping system 130 and the process repeated until all sections of the digestion region 18 have been mixed. This process can be repeated semi-continuously on a defined schedule to reduce the amount of solids settling throughout the bottom of the digestion region. For the sake of economy, such mixing can be carried out using a single mixing system 150. Alternatively, such process can be carried out using more than one mixing system 150, with each mixing system 150 connected at a respective piping system 130.

Another system component in the form of a solids removal system 170 in accordance with an example embodiment of the disclosed concept is shown in FIG. 6 connected with a respective piping system 130 of the plurality of piping systems 130. The solids removal system 170 comprises the mobile chopper pump 152 (previously discussed) having the suction inlet in fluid communication with the first end 133A of the first conduit defined by the first piping arrangement 132 of the respective piping system 130 of the plurality of piping systems 130 and a discharge outlet. The solids removal system 170 further comprises a solids separation system 172. As used herein, a “solids separation system” is a system that is designed to separate particular solid materials from a solid/liquid mixture, typically based on particle size and bulk density of the solid material. In the context of feedstock for anaerobic digesters, these solids often include substances such as sand, grit and gravel that are undesirable to the digestion process.

Continuing to refer to FIG. 6, the solids separation system 172 includes an inlet in fluid communication with the discharge outlet of the mobile chopper pump 152 and an outlet in fluid communication with the first end 135A of the second conduit 135 defined by the second piping arrangement 134 of the respective piping system 130. In such example, the connections between the mobile chopper pump 152, the solids separation system, and the first ends 133A and 135A are made via flexible hoses 174, 176 and 178, as well as isolation valves 158 and 160. Arrows F show the direction of flow within the piping system 130 of such example. Once the chopper pump 152 and the solids separation system 172 are connected, the isolation valves 158 and 160 can be opened and the chopper pump 152 turned on. The pump suction pulls the settled solids slurry from the bottom of the digestion region 18 and discharges it to the inlet of the mobile solids separation system 172. As the solids slurry passes through the solids separation system 172, the bulk solids are separated, discharged from the solids separation system 172 and accumulated in a pile 180 for disposal. The clean liquid exits the solids separation system 172 and is returned to the upper part of the digestion region 18 through the second conduit 135 via the flexible hose 178. Once the chopper pump 152 has been running for a certain period, it can be disconnected and moved to the next piping system 130 and the process repeated until solids have been sufficiently removed from each section of the digestion region 18. This process can be repeated semi-continuously on a defined schedule to reduce the amount of settled solids at the bottom of the digestion region 18. For the sake of economy, such solids removal can be carried out using a single solids removal system 170. Alternatively, such process can be carried out using more multiple solids removal systems 170, with each connected at a respective piping system 130. Additionally, mixing and solids removal can be carried out simultaneously by employing both the mixing system 150 and the solids removal system 170 simultaneously at different piping systems 130.

From the foregoing examples it is thus to be readily appreciated that embodiments of the disclosed concept provide solutions for maintaining racetrack digesters in operating condition that improve general operation thereof and reduce the need/frequency for costly cleanouts. It is to be appreciated that the example embodiments/components described herein may be varied depending on the particular application without varying from the scope of the disclosed concept.

While specific embodiments of the disclosed concept have been described in detail, it will be appreciated by those skilled in the art that various modifications and alternatives to those details could be developed in light of the overall teachings of the disclosure. Accordingly, the particular arrangements disclosed are meant to be illustrative only and not limiting as to the scope of disclosed concept which is to be given the full breadth of the claims appended and any and all equivalents thereof.

In the claims, any reference signs placed between parentheses shall not be construed as limiting the claim. The word “comprising” or “including” does not exclude the presence of elements or steps other than those listed in a claim. In any device claim enumerating several means, several of these means may be embodied by one and the same item of hardware. The word “a” or “an” preceding an element does not exclude the presence of a plurality of such elements. The mere fact that certain elements are recited in mutually different dependent claims does not indicate that these elements cannot be used in combination.

What is claimed is:

1. A racetrack digester for use in obtaining biogas from organic materials through the process of anaerobic digestion, the racetrack digester comprising:
a containment wall disposed about and defining a digestion region structured to retain a volume of the organic materials;
a plurality of mixer arrangements spaced along the containment wall, each mixer arrangement structured to move the organic materials within the digestion region and comprising:
a rotatable shaft that extends into the digestion region;
a propeller coupled to a distal end of the shaft in the digestion region; and
a drive arrangement coupled to a proximal end of the shaft opposite the propeller, the drive arrangement structured to rotate the shaft and propeller to move the organic materials about the digestion region;
a plurality of piping systems spaced along the containment wall, each piping system comprising:
a first piping arrangement defining a first conduit extending between a first end positioned at or about a top of the containment wall outside of the digestion region and a second end positioned at or about a floor of the containment region; and
a second piping arrangement defining a second conduit extending between a first end positioned at or about a top of the containment wall outside of the digestion region and a second end positioned at or near a normal liquid level within the digestion region; and
a system component associated with a respective piping system of the plurality of piping systems, wherein the system component comprises one of:
a mixing system comprising a mobile chopper pump having a suction inlet in fluid communication with the first end of the second conduit defined by the second piping arrangement of the respective piping system of the plurality of piping systems and a discharge outlet in fluid communication with the first end of the first conduit defined by the first piping arrangement of the respective piping system; or
a solids removal system comprising:
a mobile chopper pump having a suction inlet in fluid communication with the first end of the first conduit defined by the first piping arrangement of the respective piping system of the plurality of piping systems and a discharge outlet; and
a solids separation system having an inlet in fluid communication with the discharge outlet of the mobile chopper pump and an outlet in fluid communication with the first end of the second conduit defined by the second piping arrangement of the respective piping system.

2. The racetrack digester of claim 1, wherein each piping system of the plurality of piping systems further comprises a first isolation valve coupled at the first end of the first conduit and a second isolation valve coupled at the first end of the second conduit,
wherein the first isolation valve is positioned and structured to selectively control passage of fluid through the first conduit, and
wherein the second isolation valve is positioned and structured to selectively control passage of fluid through the second conduit.

3. The racetrack digester of claim 1, wherein the plurality of mixer arrangements and the plurality of piping systems are spaced along the containment wall such that at least one piping system is disposed between any two adjacent mixer arrangements.

4. The racetrack digester of claim 1, wherein the plurality of mixer arrangements and the plurality of piping systems are spaced along the containment wall such that at least two piping systems of the plurality of piping systems are disposed between at least two adjacent mixer arrangements of the plurality of mixer arrangements.

5. The racetrack digester of claim 1, wherein the first piping arrangement comprises:
a straight first portion that extends through the containment wall;
a straight end portion positioned near the floor of the digestion region; and
a straight intermediate portion that extends between the first portion and the end portion and is coupled with each of the first portion and the end portion via elbows.

6. The racetrack digester of claim 5, wherein the elbows comprise forty five degree elbows.

7. The racetrack digester of claim 1, wherein the second piping arrangement comprises a single straight portion that extends through the containment wall.

8. The racetrack digester of claim 5, wherein the second piping arrangement comprises a single straight portion that extends through the containment wall.

9. The racetrack digester of claim 1, wherein the plurality of mixer arrangements comprises at least seven mixer arrangements, and wherein the plurality of piping systems comprises at least eight piping systems.

10. The racetrack digester of claim 1, further comprising another system component associated with another respective piping system of the plurality of piping systems.

11. The racetrack digester of claim 10, wherein the system component comprises the mixing system and the other system component comprises another mixing system.

12. The racetrack digester of claim 10, wherein the system component comprises the mixing system and the other system component comprises the solids removal system.

13. The racetrack digester of claim 1, wherein each mixer arrangement is disposed in a fixed position along the containment wall and wherein the rotatable shaft of each mixer arrangement extends through the containment wall.
